(19) United States
(12) Patent Application Publication
(10) Pub. No.: US 2025/0090329 A1
(43) Pub. Date: Mar. 20, 2025

(54) IMPLANTS FOR ARTICULAR CARTILAGE REPAIR

(71) Applicant: Shu-Tung and Alice Li Foundation Inc., Oakland, NJ (US)

(72) Inventor: Shu-Tung Li, Franklin Lakes, NJ (US)

(73) Assignee: Shu-Tung and Alice Li Foundation Inc., Oakland, NJ (US)

(21) Appl. No.: 18/882,107

(22) Filed: Sep. 11, 2024

Related U.S. Application Data
(60) Provisional application No. 63/582,691, filed on Sep. 14, 2023.

(51) Int. Cl. A61F 2/30

(52) U.S. Cl. CPC A61F 2/30756; A61F 2002/30062

(57) ABSTRACT

This disclosure describes implants for the repair and regeneration of articular cartilage that includes stratified multi-layered structure zones that simulate the extracellular matrix of the native articular cartilage. The various zones are physically and mechanically integrated for in vivo stability. Methods for fabricating the implants and methods of use thereof are also disclosed.

### CROSS-REFERENCE TO RELATED APPLICATIONS

This application claims the benefit under 35 U.S.C. § 119 (e) of the earlier filing date of U.S. Provisional Patent No. 63/582,691 filed on Sep. 14, 2023, which is hereby incorporated by reference in its entirety.

### FIELD OF THE INVENTION

This invention relates generally to implants for articular cartilage repair.

### BACKGROUND OF THE INVENTION

Articular cartilage covers the ends of all bones that form the articulating joints in humans and animals. In the joint, articular cartilage functions to distribute force, and also serves as a lubricant in the area of contact between the bones. Injury to the articular cartilage, either from sports (acute injury) or due to degeneration of the articular cartilage (chronic injury) from aging or overuse of the joint, can eventually lead to unavoidable osteoarthritis of the joint if left untreated. A joint lacking articular cartilage is subjected to stress concentration and friction, thereby preventing ease of motion. Thus, loss of articular cartilage typically leads to decreased joint motion and painful arthritis. Since articular cartilage is an avascular tissue, when damaged either from trauma or disease, it does not repair itself. Currently, methods for clinical repair of damaged articular cartilage are limited to the following three modalities: microfracture, mosaicplasty, and matrix-induced autologous chondrocyte implant (MACI).

Microfracture is a simple arthroscopic procedure and by far the most common method used as a first-line treatment for symptomatic chondral defect. The method relies on creating microfractures in the subchondral bone which results in diffusion of blood and of bone marrow cells/growth factors into the microfractures, thereby promoting healing.

Mosaicplasty involves harvest of multiple cylindrical osteochondral plugs from low-weight-bearing areas within the knee joint and their subsequent transplantation to a chondral defect to create a mosaic pattern.

Historically, an autologous chondrocyte transplantation implant (ACI) was applied to treat articular cartilage defects where a small piece of cartilage is harvested from a low weight-bearing area of the knee joint. The chondrocytes in the cartilage fragment are isolated and expanded in culture to reach a desired number of chondrocytes. After 4 to 6 weeks, the joint is opened, and the defect is covered with a periosteal patch obtained from the upper tibial surface. The chondrocyte suspension is then injected underneath this patch. The tissue regenerated from this approach is fibrocartilage or hyaline cartilage or a combination of the two.

None of the above-mentioned treatment modalities is ideal. First, although the procedure is simple, the microfracture technique does not regrow the cartilage with the right tissue composition, i.e., fibrocartilage instead of articular cartilage. The regenerated fibrocartilage does not meet the functional requirement of the articular cartilage, and consequently, it does not last long in the repaired site in vivo. However, in the absence of any good solutions, it does provide a quick fix to the problem of pain and return of normal physical activity to the patients for 2-3 years.

Mosaicplasty procedure has its intrinsic deficiencies. The surface geometry of the harvested autograft often does not fit with the injured or diseased tissues. This could be one of the reasons that the tissue at the interface between the autograft and the adjacent host tissue does not heal well. The source availability of the autologous tissue is also a problem, not to mention the donor site morbidity complications.

One of the main drawbacks of the ACI-based technology is the cell harvest and subsequent cell expansion technology steps. This two-stage procedure has created quite a limitation of the surgery. Because of the additional cell culture, the outcomes of the procedure are often not predictable.

Thus, there is a strong need for developing an improved biocompatible and bioresorbable implant for the repair and regeneration of articular cartilage.

### SUMMARY OF THE INVENTION

This disclosure provides a biocompatible and bioresorbable implant for articular cartilage repair, which eliminates or reduces the disadvantages and problems associated with currently available methods of treatment.

In one aspect, this disclosure provides a biocompatible and bioresorbable extracellular matrix (ECM) implant having physically and mechanically stable stratified multi-phasic structural zones. In some embodiments, the stable stratified multi-phasic structural zones comprise: (i) a surface zone of type II and type I collagen fibers aligned parallel to surface of the ECM implant, wherein the surface zone has a thickness from about 0.05 mm to about 0.5 mm; (ii) a second zone of randomly oriented type II collagen fibers, wherein the second zone has a thickness from about 1.5 mm to about 2.0 mm; (iii) a third zone of type II collagen fibers oriented perpendicular to surface of the ECM implant, wherein the third zone has a thickness from about 1.0 mm to about 1.5 mm; (iv) a fourth zone of mineralizing cartilage comprising partially oriented type II collagen fibers and randomly oriented type I collagen fibers in combination with calcium-based mineral particles, wherein the fourth zone has a thickness of about 0.25 mm to about 0.75 mm; and (v) a mineralized zone comprising randomly oriented type I collagen fibers and calcium-based mineral particles, wherein the mineralized zone has a thickness from about 2.0 mm to about 4.0 mm.

In some embodiments, the implant is adapted for repairing large articular cartilage defects, and has an area ranging from about 3 cm² to about 12 cm² and a thickness from about 0.6 mm to about 1 cm.

In some embodiments, the implant is adapted for treating a small injury of an articular cartilage, and wherein the stratified multi-phasic structural zones are confined within a cell permeable biopolymeric tubular delivery vehicle having a diameter from about 0.5 cm to about 1 cm and a length from about 0.6 cm to about 1 cm to assist the delivery and insertion of the implant to a repair site of the articular cartilage.

In some embodiments, the implant comprises polysaccharides. In some embodiments, the polysaccharides comprise chondroitin sulfate, keratin sulfate, hyaluronic acid, or a combination thereof.

In some embodiments, the implant comprises a type III collagen, a type V collagen, a type IX collagen, a type XI collagen, or a combination thereof.

In some embodiments, the implant comprises cells. In some embodiments, the cells comprise articular chondrocytes, mononuclear cells, stem cells, or a combination thereof.

In some embodiments, the implant comprises bioactive molecules. In some embodiments, the bioactive molecules comprise platelet-rich plasma (PRP), growth factors, fibronectin, fibromodulin, biglycan, decorin, or a combination thereof.

In some embodiments, the type II collagen fibers are mixed with the type I collagen fibers at a weight ratio of from 90:10 to 60:40. In some embodiments, type I collagen fibers or type II collagens are isolated from human or animal tissues or by genetic engineering technologies. As used herein, the term “genetic engineering technologies” refers to a process of preparing recombinant human collagen by transcribing a specific collagen gene segment and expressing in yeast, bacteria, or animal cells.

In some embodiments, the biopolymeric tubular delivery vehicle is a porous tubular biopolymer-based membrane. In some embodiments, the porous tubular biopolymer-based membrane comprises a tubular type I collagen, or a type I and type II composite collagen-based membrane.

In some embodiments, the porous tubular biopolymer-based membrane is a porous tubular type I collagen-based membrane. In some embodiments, the tubular type I collagen-based membrane has a pore size of from about 20 μm to about 500 μm. In some embodiments, the tubular type I collagen-based membrane has a wall thickness of from about 0.01 mm to about 0.5 mm.

In some embodiments, the calcium-containing mineral particles comprise synthetic and/or natural calcium-containing compounds. In some embodiments, the calcium-containing mineral particles comprise natural carbonate apatite particles.

In some embodiments, the stratified multi-phasic structural zones comprise from about 20% (w/w) to about 80% (w/w) of the calcium-containing mineral particles.

In another aspect, this disclosure provides a method of fabricating the implant for repairing large articular cartilage defects. In some embodiments, the method comprises forming physically and mechanically stable stratified multi-phasic structural zones by: (a) preparing a fifth subchondral bone zone by mixing randomly oriented type I collagen fibers and calcium-based minerals; (b) preparing a fourth calcified cartilage zone by mixing randomly oriented type I fibers with calcium-based minerals and partial vertically oriented type II collagen fibers; (c) preparing a third deep zone by orientating the extruded type II collagen fibers in the direction orthogonal to the surface of the implant; (d) preparing a second middle/transitional zone by cryo-milling purified type II collagen fibers into microfibers of random fiber orientation; (e) preparing a surface superficial zone of type II and type I collagen-fiber sheet of random fiber orientation in two dimensions; (f) stabilizing the stratified multi-phasic structural zones with type I or type II collagen gelatin; and (g) compressing the stratified multi-phasic structural zones to a fixed height to obtain an implant.

In some embodiments, the method further comprises crosslinking, rinsing, and lyophilizing the implant.

In another aspect, this disclosure also provides a method of fabricating the implant for treating a small injury of an articular cartilage, forming physically and mechanically stable stratified multi-phasic structural zones by: (a) preparing a fifth subchondral bone zone by mixing randomly oriented type I collagen fibers and calcium-based minerals; (b) preparing a fourth calcified cartilage zone by mixing randomly oriented type I fibers with calcium-based minerals and partial vertically oriented type II collagen fibers; (c) preparing a third deep zone by orientating the extruded type II collagen fibers in the direction orthogonal to the surface of the implant; (d) preparing a second middle/transitional zone by cryo-milling purified type II collagen fibers into microfibers of random fiber orientation; (e) preparing a surface superficial zone of type II and type I collagen-fiber sheet of random fiber orientation in two dimensions; (f) stabilizing the stratified multi-phasic structural zones with type I or type II collagen gelatin; (g) compressing the stratified multi-phasic structural zones to a fixed height to obtain an implant; (h) shaping the implant to a cylindrical implant of a defined diameter using a cylindrical knife cutter; and (i) inserting the cylindrical implant into a defined porous tubular collagen matrix.

In yet another aspect, this disclosure provides an implant fabricated according to the method described herein.

One aspect of this disclosure relates to a biocompatible and bioresorbable implant having a stratified physically and mechanically stable multi-phasic structure that simulates the natural ECM of the articular cartilage. The implant has a total of five structure zones, a superficial zone consisting of type II collagen fibers (or type II collagen fibers mixed with type I collagen fibers at a weight ratio such as 90:10 to 60:40) organized in parallel to the surface of the implant with a thickness from about 0.1 mm to about 0.5 mm, a middle (transitional) zone consisting of randomly oriented type II collagen fibers with a thickness from about 1 mm to about 2 mm, a deep zone consisting of vertically oriented type II collagen fibers with a thickness from about 0.5 mm to about 1.5 mm, a calcified cartilage zone consisting of vertically oriented type II collagen fibers, randomly oriented type I collagen fibers, and calcium-based mineral particles having a thickness of about 0.5 mm to about 1 mm, and a subchondral bone zone consisting of type I collagen fibers and calcium-based mineral particles with a thickness depending on the depth of the injured bone tissue to be repaired, generally ranging from about 2 mm to about 4 mm.

In one embodiment, the implant layers are organized within a defined space having a dimension of about 4 cm×3 cm×1 cm (L×W×H). The size and shape of the implant can be trimmed to fit the cartilage defect using a pair of scissors or cutting with a scalpel (FIG. 1).

In another embodiment, the layers are organized within a nutrient and cell permeable tubular matrix with a diameter from about 5 mm to about 10 mm and a length from about 6 mm to about 10 mm (FIG. 2, implant plug). The tubular matrix is engineered from biopolymeric molecules, preferably type I collagen fibers, and is designed to be resorbed within about three months to facilitate the integration of the implant with host articular cartilage tissue (FIG. 2).

The implant plug is designed to be delivered using a tubular canulae via arthroscopy or other surgical means. Multiple implant plugs can be inserted into the defect in a mosaic pattern similar to the mosaicplasty procedure using the autograft plugs described above. The delivery canulae is depicted in FIG. 3.

In yet another embodiment, the implant contains glycosaminoglycans (GAGs) in various layers. The GAGs used include chondroitin sulfate (CS), keratin sulfate (KS), and hyaluronic acid (HA). The quantity of GAGs added to each layer is about 10% in layer 1 (e.g., the surface zone), 20% in layer 2 (e.g., the second zone), and layer 5 (e.g., the mineralized zone), 30% in layer 3 (e.g., the third zone), 40% in layer 4 (e.g., the fourth zone) of the weight of collagen in each layer/zone.

In a further embodiment, bioactive materials such as cells and growth factors can be incorporated into the implant during the surgery by injecting bioactive materials into the implant. The cells include autologous articular chondrocytes and autologous or allogenic stem cells. In some embodiments, the cells include autologous mesenchymal stem cells and/or articular chondrocytes. In some embodiments, the cells may include autologous articular chondrocytes in the scaffold to facilitate wound healing. In the bioactive molecules category, TGF-β is known to promote chondrogenic differentiation of stem cells. TGF-β can be added to the implant during the surgery via injection. Human recombinant TGF-β is preferred. Since platelet-rich plasma (PRP) contains a pool of bioactive molecules, including TGF-β, autologous PRP can be harvested from the blood of the patient during the surgery and added to the implant to enhance the healing and regeneration of the new cartilage tissue.

In use, a large sheet-like implant can be used for defects greater than 2 cm². The defect is first debrided and cleaned. The size of the defect is measured. The implant is then sized and trimmed to the size of the defect. Upon placement of the implant over the defect, a biocompatible membrane (e.g., autologous periosteum from the upper tibia of the patient or a biological animal tissue membrane, Chondro-Guide, marketed by Geistlich, a Swiss company), can be used to cover the implant and sutured onto the host tissue to protect the implant from shear stress induced delamination. If the injury reaches the subchondral bone, the microfracture technique can be applied to allow the blood, bone marrow, and bioactive molecules to move up to the injury site to promote healing. It is anticipated that the simulated ECM implant would influence the stem cells from the bone marrow to differentiate into the lineage of articular chondrocytes to produce the ECM of the articular cartilage instead of fibrocartilage as seen from the pure microfracture repair techniques alone. Additionally, autologous stem cells, chondrocytes, and PRP can be added via needle injection to facilitate and promote healing.

When the defect is smaller than 2 cm², then implant plugs can be applied. Here, the implant plugs can be implanted according to those described in FIG. 3.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 shows an example implant for large defect treatment.

FIG. 2 shows an example implant for the treatment of small articular cartilage defects.

FIG. 3 shows the vehicle for the delivery of an articular cartilage repair implant for the treatment of small defects.

FIG. 4 shows chondrocyte proliferation in multi-layer matrices over 7 days.

FIGS. 5A, 5B, and 5C show representative stress vs. strain curves obtained after unconfined compression (FIG. 5A) and shear (FIG. 5B) testing of the multi-layer matrices are shown. From these curves, the average compressive modulus at 10%, 20%, and 50% strain as well as the average shear modulus was obtained (FIG. 5C). Samples were hydrated in PBS for 1 hour at room temp prior to testing at room temp.

### DETAILED DESCRIPTION OF THE INVENTION

This disclosure provides a biocompatible and bioresorbable implant for articular cartilage repair, which eliminates or reduces the disadvantages and problems associated with currently available methods of treatment.

In one aspect, this disclosure provides a biocompatible and bioresorbable extracellular matrix (ECM) implant having physically and mechanically stable stratified multi-phasic structural zones. In some embodiments, the stable stratified multi-phasic structural zones comprise a surface zone of type II and type I collagen fibers aligned parallel to the surface of the ECM implant. In some embodiments, the surface zone has a thickness from about 0.05 mm to about 0.5 mm (e.g., 0.05 mm, 0.06 mm, 0.07 mm, 0.08 mm, 0.09 mm, 0.1 mm, 0.11 mm, 0.12 mm, 0.13 mm, 0.14 mm, 0.15 mm, 0.16 mm, 0.17 mm, 0.18 mm, 0.19 mm, 0.2 mm, 0.21 mm, 0.22 mm, 0.23 mm, 0.24 mm, 0.25 mm, 0.26 mm, 0.27 mm, 0.28 mm, 0.29 mm, 0.3 mm, 0.31 mm, 0.32 mm, 0.33 mm, 0.34 mm, 0.35 mm, 0.36 mm, 0.37 mm, 0.38 mm, 0.39 mm, 0.4 mm, 0.41 mm, 0.42 mm, 0.43 mm, 0.44 mm, 0.45 mm, 0.46 mm, 0.47 mm, 0.48 mm, 0.49 mm, 0.5 mm, or any intermediate values therebetween).

In some embodiments, the stable stratified multi-phasic structural zones comprise a second zone of randomly oriented type II collagen fibers. In some embodiments, the second zone has a thickness from about 1.5 mm to about 2.0 mm (e.g., 1.5 mm, 1.51 mm, 1.52 mm, 1.53 mm, 1.54 mm, 1.55 mm, 1.56 mm, 1.57 mm, 1.58 mm, 1.59 mm, 1.6 mm, 1.61 mm, 1.62 mm, 1.63 mm, 1.64 mm, 1.65 mm, 1.66 mm, 1.67 mm, 1.68 mm, 1.69 mm, 1.7 mm, 1.71 mm, 1.72 mm, 1.73 mm, 1.74 mm, 1.75 mm, 1.76 mm, 1.77 mm, 1.78 mm, 1.79 mm, 1.8 mm, 1.81 mm, 1.82 mm, 1.83 mm, 1.84 mm, 1.85 mm, 1.86 mm, 1.87 mm, 1.88 mm, 1.89 mm, 1.9 mm, 1.91 mm, 1.92 mm, 1.93 mm, 1.94 mm, 1.95 mm, 1.96 mm, 1.97 mm, 1.98 mm, 1.99 mm, 2 mm, or any intermediate values therebetween).

In some embodiments, the stable stratified multi-phasic structural zones comprise a third zone of type II collagen fibers oriented perpendicular to surface of the ECM implant. In some embodiments, the third zone has a thickness from about 1.0 mm to about 1.5 mm (e.g., 1 mm, 1.01 mm, 1.02 mm, 1.03 mm, 1.04 mm, 1.05 mm, 1.06 mm, 1.07 mm, 1.08 mm, 1.09 mm, 1.1 mm, 1.11 mm, 1.12 mm, 1.13 mm, 1.14 mm, 1.15 mm, 1.16 mm, 1.17 mm, 1.18 mm, 1.19 mm, 1.2 mm, 1.21 mm, 1.22 mm, 1.23 mm, 1.24 mm, 1.25 mm, 1.26 mm, 1.27 mm, 1.28 mm, 1.29 mm, 1.3 mm, 1.31 mm, 1.32 mm, 1.33 mm, 1.34 mm, 1.35 mm, 1.36 mm, 1.37 mm, 1.38 mm, 1.39 mm, 1.4 mm, 1.41 mm, 1.42 mm, 1.43 mm, 1.44 mm, 1.45 mm, 1.46 mm, 1.47 mm, 1.48 mm, 1.49 mm, 1.5 mm, or any intermediate values therebetween).

In some embodiments, the stable stratified multi-phasic structural zones comprise a fourth zone of mineralizing cartilage comprising partially oriented type II collagen fibers and randomly oriented type I collagen fibers in combination with calcium-based mineral particles. In some embodiments, the fourth zone has a thickness of about 0.25 mm to about 0.75 mm (e.g., 0.25 mm, 0.26 mm, 0.27 mm, 0.28 mm, 0.29 mm, 0.3 mm, 0.31 mm, 0.32 mm, 0.33 mm, 0.34 mm, 0.35 mm, 0.36 mm, 0.37 mm, 0.38 mm, 0.39 mm, 0.4 mm, 0.41 mm, 0.42 mm, 0.43 mm, 0.44 mm, 0.45 mm, 0.46 mm, 0.47 mm, 0.48 mm, 0.49 mm, 0.5 mm, 0.51 mm, 0.52 mm, 0.53 mm, 0.54 mm, 0.55 mm, 0.56 mm, 0.57 mm, 0.58 mm, 0.59 mm, 0.6 mm, 0.61 mm, 0.62 mm, 0.63 mm, 0.64 mm, 0.65 mm, 0.66 mm, 0.67 mm, 0.68 mm, 0.69 mm, 0.7 mm, 0.71 mm, 0.72 mm, 0.73 mm, 0.74 mm, 0.75 mm, or any intermediate values therebetween).

In some embodiments, the stable stratified multi-phasic structural zones comprise a mineralized zone comprising randomly oriented type I collagen fibers and calcium-based mineral particles. In some embodiments, the mineralized zone has a thickness from about 2.0 mm to about 4.0 mm (e.g., 2 mm, 2.05 mm, 2.1 mm, 2.15 mm, 2.2 mm, 2.25 mm, 2.3 mm, 2.35 mm, 2.4 mm, 2.45 mm, 2.5 mm, 2.55 mm, 2.6 mm, 2.65 mm, 2.7 mm, 2.75 mm, 2.8 mm, 2.85 mm, 2.9 mm, 2.95 mm, 3 mm, 3.05 mm, 3.1 mm, 3.15 mm, 3.2 mm, 3.25 mm, 3.3 mm, 3.35 mm, 3.4 mm, 3.45 mm, 3.5 mm, 3.55 mm, 3.6 mm, 3.65 mm, 3.7 mm, 3.75 mm, 3.8 mm, 3.85 mm, 3.9 mm, 3.95 mm, 4 mm, or any intermediate values therebetween).

Similar to the intact tissue, the implant contains five separated but mechanically integrated zones, i.e., a superficial zone containing type II collagen fibers organized in a plane parallel to the surface of the articular tissue, having a thickness from about 0.05 mm to about 0.5 mm (e.g., 0.05 mm, 0.06 mm, 0.07 mm, 0.08 mm, 0.09 mm, 0.1 mm, 0.11 mm, 0.12 mm, 0.13 mm, 0.14 mm, 0.15 mm, 0.16 mm, 0.17 mm, 0.18 mm, 0.19 mm, 0.2 mm, 0.21 mm, 0.22 mm, 0.23 mm, 0.24 mm, 0.25 mm, 0.26 mm, 0.27 mm, 0.28 mm, 0.29 mm, 0.3 mm, 0.31 mm, 0.32 mm, 0.33 mm, 0.34 mm, 0.35 mm, 0.36 mm, 0.37 mm, 0.38 mm, 0.39 mm, 0.4 mm, 0.41 mm, 0.42 mm, 0.43 mm, 0.44 mm, 0.45 mm, 0.46 mm, 0.47 mm, 0.48 mm, 0.49 mm, 0.5 mm, or any intermediate values therebetween). Example 4 describes additional details of preparation of the superficial zone.

Below the superficial zone lies a thick middle (transitional) zone of randomly packed type II collagen fibers with a height of from about 1.5 mm to about 2.0 mm (e.g., 1.5 mm, 1.51 mm, 1.52 mm, 1.53 mm, 1.54 mm, 1.55 mm, 1.56 mm, 1.57 mm, 1.58 mm, 1.59 mm, 1.6 mm, 1.61 mm, 1.62 mm, 1.63 mm, 1.64 mm, 1.65 mm, 1.66 mm, 1.67 mm, 1.68 mm, 1.69 mm, 1.7 mm, 1.71 mm, 1.72 mm, 1.73 mm, 1.74 mm, 1.75 mm, 1.76 mm, 1.77 mm, 1.78 mm, 1.79 mm, 1.8 mm, 1.81 mm, 1.82 mm, 1.83 mm, 1.84 mm, 1.85 mm, 1.86 mm, 1.87 mm, 1.88 mm, 1.89 mm, 1.9 mm, 1.91 mm, 1.92 mm, 1.93 mm, 1.94 mm, 1.95 mm, 1.96 mm, 1.97 mm, 1.98 mm, 1.99 mm, 2 mm, or any intermediate values therebetween). The type II collagen fibers in the middle zone are in the form of randomly distributed micro-fibers with a size in the range of from 50 μm to about 75 μm. The middle zone can be constructed either by 3D printing or by molding of the microfibers. Example 4 describes additional details of preparation of the middle zone. GAGs can be mixed with type II fibers in the form of solid fibers or particles. The weight ratio of type II collagen to GAGs is about 80:20.

Beneath the middle zone is the deep zone of vertically packed type II collagen fibers with a height from 1.0 mm to about 1.5 mm (e.g., 1 mm, 1.01 mm, 1.02 mm, 1.03 mm, 1.04 mm, 1.05 mm, 1.06 mm, 1.07 mm, 1.08 mm, 1.09 mm, 1.1 mm, 1.11 mm, 1.12 mm, 1.13 mm, 1.14 mm, 1.15 mm, 1.16 mm, 1.17 mm, 1.18 mm, 1.19 mm, 1.2 mm, 1.21 mm, 1.22 mm, 1.23 mm, 1.24 mm, 1.25 mm, 1.26 mm, 1.27 mm, 1.28 mm, 1.29 mm, 1.3 mm, 1.31 mm, 1.32 mm, 1.33 mm, 1.34 mm, 1.35 mm, 1.36 mm, 1.37 mm, 1.38 mm, 1.39 mm, 1.4 mm, 1.41 mm, 1.42 mm, 1.43 mm, 1.44 mm, 1.45 mm, 1.46 mm, 1.47 mm, 1.48 mm, 1.49 mm, 1.5 mm, or any intermediate values therebetween). The vertically packed type II collagen fibers require the orientation of the collagen fibers. The technique of extrusion of a thick collagen solution (>30 mg/l ml) was applied using a nozzle size of 0.5 mm to 1.2 mm into isopropanol solvent. The extruded filament can be lifted from the solvent and dried in air. The extruded fibers are aligned and are stabilized with type II collagen gelation before cutting to the length (between 1.0 mm and 1.5 mm). Example 4 describes additional details of deep zone type II collagen fibers. GAGs can be mixed with type II collagen fibers in a weight ratio of 70:30 of collagen fibers to GAGs.

The next zone beneath the deep zone is the calcified cartilage zone, where the extracellular matrix of this zone contains type I collagen fibers, type II collagen fibers, and calcium-containing minerals. In this zone in vivo, articular chondrocytes undergo atrophy and apoptosis in a process termed endochondral ossification, where atrophic chondrocytes are replaced by osteoblasts and bone. The thin layer between the deep zone and the calcified cartilage is called the tide mark. In engineering the deep zone, type II collagen fibers are directly extending from the deep zone into the calcified cartilage zone. The calcified cartilage zone is, therefore, the zone containing a mixture of type I and type II collagen fibers with a calcium containing mineral (e.g., carbonate apatite). Example 4 describes additional details of preparing the calcified cartilage zone along with the integration of type II collagen fibers from the deep zone with the calcified cartilage zone. Again, the GAGs can be mixed with collagen (type I and type II) and mineral at a weight ratio of about 60 (collagens): 10 (mineral): 30 (GAG).

Lastly, adjacent to the calcified cartilage zone is the subchondral bone which has a structure of normal cancellous (trabecular) bone. This zone contains type I collagen fibers and calcium-based mineral particles at a weight ratio of about 20 (collagen): 80 (mineral).

In some embodiments, the area of the overall ECM implant can vary from 3 cm² to about 12 cm² (e.g., 3 cm², 3.5 cm², 4 cm², 4.5 cm², 5 cm², 5.5 cm², 6 cm², 6.5 cm², 7 cm², 7.5 cm², 8 cm², 8.5 cm², 9 cm², 9.5 cm², 10 cm², 10.5 cm², 11 cm², 11.5 cm², 12 cm², or any intermediate values therebetween) for the repair of large articular defects with a thickness of the implant varies from about 5 mm to about 10 mm (e.g., 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, or any intermediate values therebetween).

In some embodiments, the ECM implant is packed within a cell and nutrient permeable cylindrical housing. The inner diameter of the cylinder is from about 5 mm to about 10 mm (e.g., 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, or any intermediate values therebetween) with a wall thickness of about 0.1-0.3 mm (e.g., 0.1 mm, 0.11 mm, 0.12 mm, 0.13 mm, 0.14 mm, 0.15 mm, 0.16 mm, 0.17 mm, 0.18 mm, 0.19 mm, 0.2 mm, 0.21 mm, 0.22 mm, 0.23 mm, 0.24 mm, 0.25 mm, 0.26 mm, 0.27 mm, 0.28 mm, 0.29 mm, 0.3 mm, or any intermediate values therebetween). The height of the cylinder is from about 5 mm to about 10 mm (e.g., 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, or any intermediate values therebetween). The cylinder can be made from any biocompatible and bioresorbable polymeric materials. In some embodiments, biopolymeric materials can be used to engineer cylindrical devices. In some embodiments, collagen-based materials, e.g., type I collagen or a combination of type I and type II collagen materials, can be used for engineering the delivery cylinders.

In yet another embodiment of the invention, the implants contain polysaccharides (generally referred to as glycosaminoglycans or GAGs) within different zones of the implant. GAGs covalently linked to a core protein, known as proteoglycan, form the major ground substance of the ECM matrix of the articular cartilage. In articular cartilage, the key polysaccharides (GAGs) include chondroitin sulfate (CS), keratin sulfate (KS), and hyaluronic acid (HA). The majority of the GAGs are CS (>85%) with less KS (˜10%) and HA (˜2-5%). It would be important to incorporate GAGs in the implant as GAG is known to contribute significantly to maintaining an adequate amount of H₂O in the ECM structure to support the biomechanical properties of the ECM matrix. GAGs contribute to about 10-15% dry weight of the articular cartilage. Its content increases from the surface layer to the calcified layer. As CS amounts to about 85% of the total GAG in articular cartilage, it would be reasonable to include CS in the first phase design to evaluate the effect of GAGs on chondrogenesis differentiation of stem cells.

In some embodiments, the implant contains cells and growth factors to enhance the healing of the wound and to accelerate the recovery of the physical function of the knee. In the cell area, both differentiated (chondrocytes) and undifferentiated cells (mesenchymal stem cells) can be added to the implant via injection technique during surgery. Autologous-based cells are preferred. Autologous stem cells are particularly preferred as the ECM matrix has a composition and 3D structure similar to the ECM of the native structure, which would most likely contain the necessary biochemical cues to induce differentiation of the stem cells to chondrogenic lineage, thereby accelerating the cartilage tissue deposition.

In the growth factors area, ample studies demonstrate the effectiveness of TGF-β induces chondrogenesis of stem cells. TGF-β certainly can be incorporated into the ECM implant along with stem cells for the reasons stated above. In some embodiments, human recombinant TGF-β (rhTGF-β) be used for clinical application.

Another source of growth factors is from human blood-based platelet-rich-plasma (PRP). PRP can be harvested from the patient during the surgery and goes through the PRP separation at the surgery room and injected into the ECM implant during surgery. PRP contains a pool of growth factors including TGF-β which facilitates the autologous stem cell differentiation into chondrocytes for enhancing the healing of articular cartilage lesions.

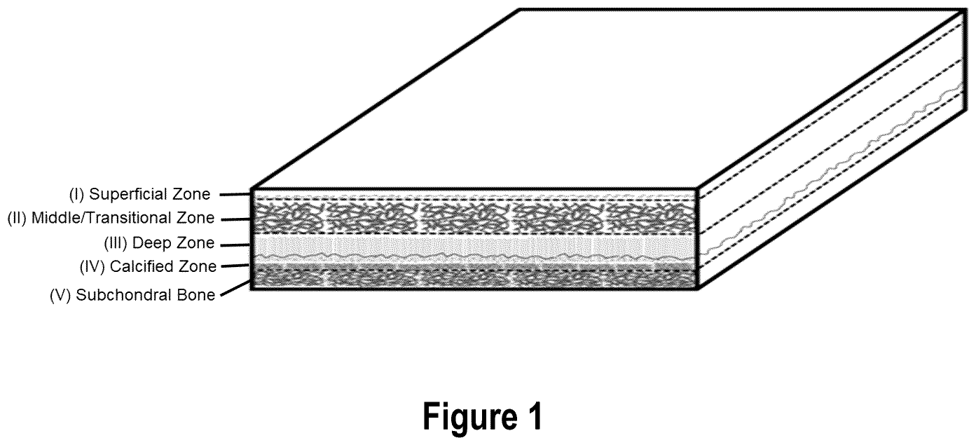

FIG. 1 depicts an example implant for large defect treatment. The implant has a dimension of about 3 cm×4 cm×1 cm (W×L×H), which includes 2-4 mm of subchondral bone layer. This size will be suitable for the repair of most large size defects of articular cartilage lesions. It can be trimmed to any size with the use of surgical scissors or a scalpel. The implant has a structure that closely mimics the ECM of articular cartilage, which can provide biochemical cues for promoting stem cell differentiation and maintaining the phenotype of articular chondrocytes.

In some embodiments, the implant is adapted for repairing large articular cartilage defects, and has an area ranging from about 3 cm² to about 12 cm² (e.g., 3 cm², 3.5 cm², 4 cm², 4.5 cm², 5 cm², 5.5 cm², 6 cm², 6.5 cm², 7 cm², 7.5 cm², 8 cm², 8.5 cm², 9 cm², 9.5 cm², 10 cm², 10.5 cm², 11 cm², 11.5 cm², 12 cm², or any intermediate values therebetween) and a thickness from about 0.6 mm to about 1 cm (e.g., 0.6 mm, 0.7 mm, 0.8 mm, 0.9 mm, 1 mm, 21 mm, 41 mm, 61 mm, 81 mm, 101 mm, 121 mm, 141 mm, 161 mm, 181 mm, 201 mm, 221 mm, 241 mm, 261 mm, 281 mm, 301 mm, 321 mm, 341 mm, 361 mm, 381 mm, 401 mm, 421 mm, 441 mm, 461 mm, 481 mm, 501 mm, 521 mm, 541 mm, 561 mm, 581 mm, 601 mm, 621 mm, 641 mm, 661 mm, 681 mm, 701 mm, 721 mm, 741 mm, 761 mm, 781 mm, 801 mm, 821 mm, 841 mm, 861 mm, 881 mm, 901 mm, 921 mm, 941 mm, 961 mm, 981 mm, 1000 mm, or any intermediate values therebetween).

In some embodiments, the implant is adapted for treating a small injury of an articular cartilage, and wherein the stratified multi-phasic structural zones are confined within a cell permeable biopolymeric tubular delivery vehicle having a diameter from about 0.5 cm to about 1 cm (e.g., 0.5 cm, 0.51 cm, 0.52 cm, 0.53 cm, 0.54 cm, 0.55 cm, 0.56 cm, 0.57 cm, 0.58 cm, 0.59 cm, 0.6 cm, 0.61 cm, 0.62 cm, 0.63 cm, 0.64 cm, 0.65 cm, 0.66 cm, 0.67 cm, 0.68 cm, 0.69 cm, 0.7 cm, 0.71 cm, 0.72 cm, 0.73 cm, 0.74 cm, 0.75 cm, 0.76 cm, 0.77 cm, 0.78 cm, 0.79 cm, 0.8 cm, 0.81 cm, 0.82 cm, 0.83 cm, 0.84 cm, 0.85 cm, 0.86 cm, 0.87 cm, 0.88 cm, 0.89 cm, 0.9 cm, 0.91 cm, 0.92 cm, 0.93 cm, 0.94 cm, 0.95 cm, 0.96 cm, 0.97 cm, 0.98 cm, 0.99 cm, 1 cm, or any intermediate values therebetween) and a length from about 0.6 cm to about 1 cm (e.g., 0.6 cm, 0.61 cm, 0.62 cm, 0.63 cm, 0.64 cm, 0.65 cm, 0.66 cm, 0.67 cm, 0.68 cm, 0.69 cm, 0.7 cm, 0.71 cm, 0.72 cm, 0.73 cm, 0.74 cm, 0.75 cm, 0.76 cm, 0.77 cm, 0.78 cm, 0.79 cm, 0.8 cm, 0.81 cm, 0.82 cm, 0.83 cm, 0.84 cm, 0.85 cm, 0.86 cm, 0.87 cm, 0.88 cm, 0.89 cm, 0.9 cm, 0.91 cm, 0.92 cm, 0.93 cm, 0.94 cm, 0.95 cm, 0.96 cm, 0.97 cm, 0.98 cm, 0.99 cm, 1 cm, or any intermediate values therebetween) to assist the delivery and insertion of the implant to a repair site of the articular cartilage.

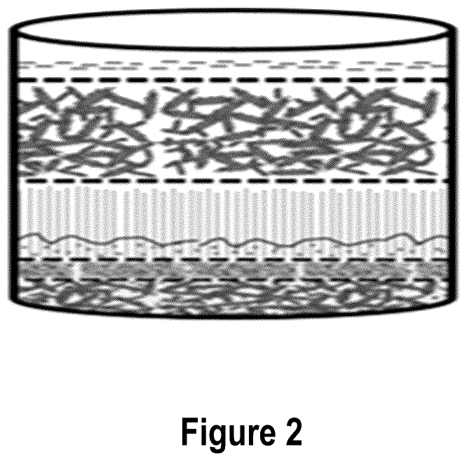

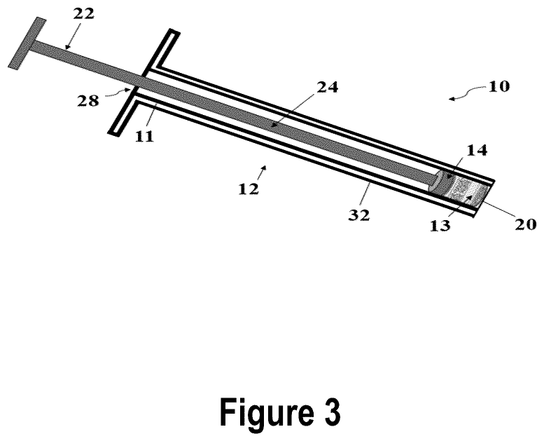

FIG. 2 depicts an example implant for the treatment of small articular cartilage defects. The implant has a diameter from about 5 mm to about 10 mm and a height of from about 6 mm to about 10 mm, which includes 2-4 mm of subchondral bone layer. The implant is confined within a tubular cell and nutrient permeable biopolymeric membrane to facilitate the delivery using a delivery vehicle which is shown in FIG. 3. The tubular membrane can be engineered from most biologic polymers and is particularly with fiber forming collagens such as type I collagen. Type I collagen fibers can be easily prepared from animal tissues such as tendons and corium of bovine. The method of engineering a tubular membrane matrix has been described in, for example, Li S T et al. (Biologic Biomaterials: Tissue derived Biomaterials (collagen), Eds. J Y Wong, JD Bronzino and DR Peterson, CRC Press (2013)), the relevant disclosure of which is herein incorporated by reference. The membrane can be perforated with a needle of defined size or with a laser beam to facilitate cell permeation and nutrient exchange.

FIG. 3 depicts the vehicle for the delivery of an articular cartilage repair implant for the treatment of small defects. Delivery system 10 includes an implant member 13 disposed within an implant delivery device 12. The primary function of delivery device 12 is to deliver implant member 13 to a desired site having a defect in the cartilage tissue of a patient and to fill the defect with the implant member 13. Delivery device 12 is made from any biocompatible material including stainless steel, synthetic polymeric materials (e.g., polyethylene, polypropylene, polyvinyl chloride, polystyrene), and other such biocompatible materials which are well known to those skilled in the art. The implant delivery device includes cannula 11 having an elongated body extending from an insertable front portion 32 to an outlet 20 at its distal end. Cannula 11 is sized to slidably receive a retaining member 22 which is introduced at a proximal end 28 of cannula 11. Flat plate 14 has an outside diameter which is slightly less than the inside diameter of insertable portion 32 to enable the rod-like member 22 to slidably move down the longitudinal axis of insertable portion 32 and maintain implant membrane 13 at outlet 20. Implant member 13 is positioned within cannula 11 to be coextensive with outlet 20 at its distal end. Retaining member 22 is slidably introduced within cannula 11 so that its flat plate contacts the proximal end of the implant member. In operation, the injury site is thoroughly cleaned and debrided to remove all the debris and diseased tissue along with the top layer of the subchondral bone. The depth of the overall defect should match the length of the implant plug such that the length of the plug matches the defect from the bone to the healthy cartilage. The delivery system 10 is then inserted into the defect site via arthroscopy or other means. While applying slight axial pressure on retaining member 22 to maintain the position of implant member 13 at the injury site, cannula 11 is withdrawn over implant member 13 and retaining member 22 until the cannula extends beyond the proximal end of implant membrane 13. This procedure can be repeated depending on the overall size of the defect. The number and the size of the implant plugs to be used are at the discretion of the surgeon.

In some embodiments, the implant comprises polysaccharides. In some embodiments, the polysaccharides comprise chondroitin sulfate, keratin sulfate, hyaluronic acid, or a combination thereof.

In some embodiments, the implant comprises a type III collagen, a type V collagen, a type IX collagen, a type XI collagen, or a combination thereof.

In some embodiments, the implant comprises cells. In some embodiments, the cells comprise articular chondrocytes, mononuclear cells, stem cells, or a combination thereof.

In some embodiments, the implant comprises bioactive molecules. In some embodiments, the bioactive molecules comprise platelet-rich plasma (PRP), growth factors, fibronectin, fibromodulin, biglycan, decorin, or a combination thereof.

In some embodiments, the type II collagen fibers mixed with the type I collagen fibers at a weight ratio of from 90:10 to 60:40 (e.g., 90:10, 88:12, 86:14, 84:16, 82:18, 80:20, 78:22, 76:24, 74:26, 72:28, 70:30, 68:32, 66:34, 64:36, 62:38, 60:40, 58:42, 56:44, 54:46, 52:48, 50:50, 48:52, 46:54, 44:56, 42:58, 40:60, or any intermediate values therebetween). In some embodiments, type I collagen fibers or type II collagens are isolated from human or animal tissues or by genetic engineering technologies.

In some embodiments, the biopolymeric tubular delivery vehicle is a porous tubular biopolymer-based membrane. In some embodiments, the porous tubular biopolymer-based membrane comprises a tubular type I collagen, or a type I and type II composite collagen-based membrane.

In some embodiments, the porous tubular biopolymer-based membrane is a porous tubular type I collagen-based membrane. In some embodiments, the tubular type I collagen-based membrane has a pore size of from about 20 μm to about 500 μm (e.g., 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or any intermediate values therebetween). In some embodiments, the tubular type I collagen-based membrane has a wall thickness of from about 0.01 mm to about 0.5 mm (e.g., 0.01 mm, 0.02 mm, 0.03 mm, 0.04 mm, 0.05 mm, 0.06 mm, 0.07 mm, 0.08 mm, 0.09 mm, 0.1 mm, 0.11 mm, 0.12 mm, 0.13 mm, 0.14 mm, 0.15 mm, 0.16 mm, 0.17 mm, 0.18 mm, 0.19 mm, 0.2 mm, 0.21 mm, 0.22 mm, 0.23 mm, 0.24 mm, 0.25 mm, 0.26 mm, 0.27 mm, 0.28 mm, 0.29 mm, 0.3 mm, 0.31 mm, 0.32 mm, 0.33 mm, 0.34 mm, 0.35 mm, 0.36 mm, 0.37 mm, 0.38 mm, 0.39 mm, 0.4 mm, 0.41 mm, 0.42 mm, 0.43 mm, 0.44 mm, 0.45 mm, 0.46 mm, 0.47 mm, 0.48 mm, 0.49 mm, 0.5 mm, or any intermediate values therebetween).

In some embodiments, the calcium-containing mineral particles comprise synthetic and/or natural calcium-containing compounds. In some embodiments, the calcium-containing mineral particles comprise natural carbonate apatite particles.

In some embodiments, the stratified multi-phasic structural zones comprise from about 20% (w/w) to about 80% (w/w) (e.g., 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), 80% (w/w), or any intermediate values therebetween) of the calcium-containing mineral particles.

In another aspect, this disclosure provides a method of fabricating the implant for repairing large articular cartilage defects. In some embodiments, the method comprises forming physically and mechanically stable stratified multi-phasic structural zones by: (a) preparing a fifth subchondral bone zone by mixing randomly oriented type I collagen fibers and calcium-based minerals; (b) preparing a fourth calcified cartilage zone by mixing randomly oriented type I fibers with calcium-based minerals and partial vertically oriented type II collagen fibers; (c) preparing a third deep zone by orientating the extruded type II collagen fibers in the direction orthogonal to the surface of the implant; (d) preparing a second middle/transitional zone by cryo-milling purified type II collagen fibers into microfibers of random fiber orientation; (e) preparing a surface superficial zone of type II and type I collagen-fiber sheet of random fiber orientation in two dimensions; (f) stabilizing the stratified multi-phasic structural zones with type I or type II collagen gelatin; and (g) compressing the stratified multi-phasic structural zones to a fixed height to obtain an implant.

In some embodiments, the method further comprises crosslinking, rinsing, and lyophilizing the implant.

In another aspect, this disclosure also provides a method of fabricating the implant for treating small injury of an articular cartilage, forming physically and mechanically stable stratified multi-phasic structural zones by: (a) preparing a fifth subchondral bone zone by mixing randomly oriented type I collagen fibers and calcium-based minerals; (b) preparing a fourth calcified cartilage zone by mixing randomly oriented type I fibers with calcium-based minerals and partial vertically oriented type II collagen fibers; (c) preparing a third deep zone by orientating the extruded type II collagen fibers in the direction orthogonal to the surface of the implant; (d) preparing a second middle/transitional zone by cryo-milling purified type II collagen fibers into microfibers of random fiber orientation; (e) preparing a surface superficial zone of type II and type I collagen-fiber sheet of random fiber orientation in two dimensions; (f) stabilizing the stratified multi-phasic structural zones with type I or type II collagen gelatin; (g) compressing the stratified multi-phasic structural zones to a fixed height to obtain an implant; (h) shaping the implant to a cylindrical implant of a defined diameter using a cylindrical knife cutter; and (i) inserting the cylindrical implant into a defined porous tubular collagen matrix.

In yet another aspect, this disclosure provides an implant fabricated according to the method described herein.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “in vitro” refers to events that occur in an artificial environment, e.g., in a test tube or reaction vessel, in cell culture, etc., rather than within a multi-cellular organism.

As used herein, “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

As used herein, “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like do not necessarily refer to the same embodiment, but may unless the context dictates otherwise.

As used herein, the terms “and/or” or “/” means any one of the items, any combination of the items, or all of the items with which this term is associated.

As used herein, the term “substantially” does not exclude “completely,” e.g., a composition which is “substantially free” from Y may be completely free from Y. Where necessary, the word “substantially” may be omitted from the definition of the present disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the invention and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. Isolation and Purification of Type I and Type II Collagen Fibers

Type II collagen fibers and type II collagen molecules were isolated from bovine articular cartilage tissue.

### Type I Collagen Fibers

Type I collagen fibers were purified similar to the method described in U.S. Pat. No. 6,391,333, the disclosure of which is incorporated herein by reference. Briefly, the fat and fascia of bovine flexor tendon were removed, and the tendon was washed in water. The cleaned tendons were then frozen and mechanically comminuted into 0.5 mm slices. The tendon slices were first extracted in purified water to remove water soluble proteins, followed by 0.5 M HCl in 0.5 M Na₂SO₄ for 24 hours, then in 0.2M NaOH in 0.5 M Na₂SO₄ for 24 hours to remove acidic and basic proteins and some lipids. Tendon slices were then extracted in isopropanol twice each for 24 hours to remove lipid moieties. The purified bovine type I collagen fibers were lyophilized and cut into small pieces with a Grindomix GM200 mechanical knife mill (Retch, Hann, NRW) and further reduced to micron size via cryo-milling using a SPEX 6775 Freezer/Mill (SPEX Sample Prep, Metuchen, NJ). The cryo-milled microfibers were sieved to collect the sizes between 50 μm to 75 μm and stored at 4° C. until use.

### Type II Collagen Fibers and Molecules

The following describes an example purification procedure for Type II collagen fibers and molecules:

Finely minced noodle-like tissue strips were frozen at −80° C. for one hour. Then, the frozen tissue was thawed at room temperature for 1 h and washed in deionized water for 30 min. This step was repeated twice for a total of 3 times during a freeze-thaw procedure. The following procedure was then performed at 4° C.

About 10 g of freeze-thawed tissue was placed in a beaker containing 1000 ml of 0.5 M NH₄Cl, pH 7 solution and kept the solution under stirring for 24 hours. The solution was replaced with a new 1000 ml of precooled 0.5M NH₄Cl (pH 7) solution and kept the solution under stirring for another 24 hours.

The NH₄Cl (pH 7) solution was removed, and tissue was washed in 500 ml water twice each for 2 hr. The water was replaced with 1000 ml precooled 0.2M NaOH solution, and the beaker was kept under constant stirring for 18 hours. The NaOH solution was replaced with a new 1000 ml precooled 0.2M NaOH solution. The new NaOH solution was under constant stirring for 24 hours.

The pH of the NaOH solution was adjusted to 7 by adding 3M HCl and kept stirring for 60 min followed by washing 2 times with 500 ml of 1% NaCl, 30 min each and then with 500 ml water for 30 min.

Next, 500 ml of 0.5M HCl was added to the tissue and kept stirring the solution for 24 hours. followed by adjusting the pH of HCl to 7 by adding 3M NaOH and kept stirring for 60 min, then washed 2 times with 500 ml 1% NaCl, 30 min each.

Then 500 ml 4% EDTA at pH 7.5 was added to the tissue for 20 hours under constant stirring followed by washing with 500 ml 1% NaCl each for 3 times, followed by washing with 300 ml water 3 times and adjusting pH to 7 with NaOH or HCl if needed.

Next, the tissue was treated with 300 ml of 4M Guanidine-HCl under constant stirring for 24 hours. After 24 hours, tissue was washed with 500 ml of water 3 times for 60 min each. Finally, the tissue was defatted with 400 ml of isopropanol twice each for 18 hours. and washed with 500 ml water twice and lyophilized.

### Type II Collagen Molecules

Purified type II collagen fibers prepared above were subjected to a pepsin treatment at room temperature to release type II collagen molecules (atelocollagen) from the purified insoluble type II collagen fibers. The solubilized type II collagen molecules were dialyzed to remove the acid, and then lyophilized and stored at 4° C.

Alternatively, the raw bovine articular cartilage strips were cryo-milled and treated directly with pepsin in 0.5M acetic acid for 24 hours at room temperature to release type II atelocollagen molecules from the tissue. The residual materials were removed via filtration. This procedure was repeated twice to remove additional type II collagen from the raw cartilage tissue. The solubilized atelocollagen molecules were purified by the differential salt precipitation techniques described by Miller and Rhodes (Methods in Enzymology Vol 82, Part A, Preparation, and characterization of the different types of collagens, p.33-64, 1982). The final type II collagen molecules were dialyzed with 0.001M acetic acid solution, lyophilized, and stored at 4° C.

### Characterization of Type I and Type II Collagen Fibers and Molecules

SDS-PAGE was used to confirm the native structure of the type I and type II collagen molecules (type I insoluble collagen and type II soluble and insoluble collagen). SDS-PAGE analysis was performed using Mini-PROTEIN Tera Cell (Cat #1658004, Bio-Rad System) and precast gel and a PowerPac Basic Supply (Cat #1645050, Bio-Rad).

Uronic acid analysis was conducted to confirm the removal of the glycosaminoglycans from insoluble type II collagen fibers. Uronic acid determination was developed based on Bitter and Muir (Bitter, T and Muir, H M, 1962 A modified uronic acid carbazole reaction, Anal. Biochem. 4:330-334).

DNA analysis was conducted to confirm the removal of cell related moieties from insoluble type I and type II collagen fibers. DNA content determination was conducted according to DNeasy Blood & Tissue kit instruction manual from Qiagen (Cat #69504) and General DNA Quantification Kit instruction manual from Abcam (Cat #ab156902).

Hydroxyproline is a unique amino acid of collagen. In general, the hydroxyproline content of fiber forming collagen is in the range of 13-14% by weight. Hydroxyproline content was determined based on Bergman and Loxley (Bergman, I and Loxley, 1963, 12:1961-1965).

Hydrothermal stability (Tₛ) was determined to evaluate the intactness of the type II collagen fibers. Tₛ was determined from the differential scanning calorimeter (DSC) (Mettler, Swiss).

TABLE 1
Test results of the type I and type II collagen fibers.

| | Type I | Type II |
|---|---|---|
| | collagen fibers | collagen fibers |
| Uronic Acid (%) | <0.5 | <0.6 |
| SDS-PAGE (% type I and type II | >97 | >97 |
| collagens based on density of the | | |
| band analysis) | | |
| Hydroxyproline content (%) | >12.5 | >12.5 |
| DNA content (ng/mg) | <50 | <50 |
| Other collagens and GAGs (%) | <3 | <3 |
| Hydrothermal stability (° C. at | >50 | >50 |
| 5° C./min speed) | | |

### Example 2. Preparation of Randomly Oriented Type I and Type II Collagen Fibers

Randomly oriented type I and type II collagen fibers were prepared directly by cryo-milling the purified fibers into microfibers using SPEX 6775 Freezer/Mill (SPEX Sample Prep, Metuchen, NJ). The range of microfiber sizes can be separated using a sieve. Microfibers in the range of 50-75 μm are adequate to function as randomly oriented collagen fibers.

### Example 3. Method of Preparing Calcium-Based Mineral Particles

Natural porous carbonate apatite was used as the calcium-based mineral for this application. Carbonate apatite was isolated from the subchondral bone of the femoral head of bovine and processed as described below. The cancellous bone of the femoral heads was supplied from a local abattoir and thoroughly washed in water, and all adhering soft tissues were removed. The bone was dried and ground into small particles. The particles were deproteinized at about 500° C. for 24 to 48 hours to remove organic materials. The mineral particles were then ground into micro-sized particles. The particles having a size range between 50-75 μm were collected and stored at −4° C. until use.

### Example 4. Method of Engineering Collagen-Based ECM Implant for Large Articular Cartilage Defect Repair

### 4.1. Method of Preparing Superficial Zone of the ECM Implant (See FIG. 1)

The superficial zone contains type II collagen fibers mixed with a lesser amount of type I collagen fibers arranged along the surface of the ECM scaffold implant. A type I collagen dispersion was first prepared by swelling 0.6 mg of purified type I collagen fibers in 100 ml of 0.6 M lactic acid solution overnight. The swollen fibers were then homogenized to produce a type I collagen fibril dispersion. A fixed amount of insoluble type II collagen microfibers was then added to the type I collagen dispersion to achieve a weight ratio 60:40 of type II to type I collagen and mixed the two collagens using a stir bar. The pH of the mixture was brought to pH about 5 using 1 N NH₄OH to coacervate the type I collagen fibers. Next, the composite mixture was poured into a square mold (32×32×10 mm) and a compression block was placed on top for one hour at room temperature to dehydrate the coacervated fibers. The dehydrated matrix was then lyophilized, and the porous sponge was humidified and further compressed between two Teflon blocks to a target thickness of 0.10 to 0.13 mm. The compressed composite dense sponge was kept in the freezer until use.

### 4.2. Method of Preparing Middle (Transitional) Zone of the ECM Implant

The middle (translational) zone contains randomly oriented type II collagen fibers for a total thickness in the range from 1.5 mm to about 2 mm. The randomly oriented type II collagen fibers were prepared by cryo-milling the purified collagen fibers using a SPEX 6775 Freezer/Mill (SPEX Sample Prep, Metuchen, NJ). The microfibers were filtered using stainless steel sieves (Cole-Parmer US, Vernon Hills, IL) to produce different sizes of microfibers. The microfiber size ranging from 50-75 μm was selected for the middle/transitional zone engineering.

### 4.3. Method of Preparing Deep Zone of the ECM Implant

In the deep zone, type II collagen fibers are oriented perpendicularly to the surface. To engineer the oriented fibers in one direction, the extrusion technique was applied to orient the type II collagen molecules using a syringe pump to control the flow of the viscous liquid with a nozzle diameter of 1.2 mm. The oriented molecules were extruded into an isopropanol bath to exchange the water with isopropanol. The aggregated fibers were lifted from the solution and dried in air. The dried fibers were lightly crosslinked with HCHO vapor, rinsed with water to remove the HCHO residuals, and dried again. Finally, the fibers were dipped in a type II collagen gelatin solution (heat denatured type II collagen) to bind the fibers together, lyophilized, and cut into 1.5 mm thickness using a scalpel.

### 4.4. Method of Preparing the Calcified Cartilage Zone of the ECM Implant

The calcified cartilage zone contains a mixture of type II collagen fibers, type I collagen fibers and calcium-containing mineral molecules. These materials were prepared above and were sampled together to constitute the calcified cartilage zone at a weight ratio of about 50% type II collagen extended from the deep zone, 30% type I collagen fibers in the microfiber form, and 20% carbonate apatite mineral particles (50-75 μm) of the zonal materials.

### 4.5. Method of Preparing Subchondral Bone Zone of the ECM Implant

The subchondral bone zone consisted of type I collagen fibers and carbonate apatite mineral particles. The weight ratio of these materials is about 80% of minerals (50-75 μm) and 20% of type I collagen microfibers (50-75 μm).

### 4.6. Method of Assembling the Overall ECM Implant for Large Defect

The overall type II collagen-based ECM implant was engineered from the bottom up, i.e., from the subchondral bone zone to the superficial zone. The mixture of calcium-based mineral (carbonate apatite) with type I collagen fibers (80:20 (w:w)) from section Example 4.5 was first placed at the bottom of the mold having a fixed area and scaled height. The mineral-collagen mixture was lightly hydrated to form a paste-like morphology. A type II collagen gelatin glue was then spread on the top of the bone layer. The calcified cartilage zone was layered onto the surface of the bone zone. Again, a layer of type II collagen glue was spread on the top of the layer. While the glue was still wet, the vertically aligned type II collagen fibers from the deep zone were placed over the calcified cartilage zone. A vertical force was applied to ensure that the fibers penetrated the calcified cartilage zone. Again, the surface of the deep zone was wetted with type II collagen gelatin, the randomly oriented type II collagen fibers were then placed on the surface of the deep zone to secure the layer structure. With the help of type II collagen gelatin, the superficial layer membrane was glued to the underneath middle/transitional zone to complete the ECM implant. The final implant was then lightly hydrated in the mold and compressed to the desired height using a weight. The compressed scaffold was then lyophilized and crosslinked with 1-ethyl-3-(3-dimethylamino propyl) carbodiimide (EDC) in 75% propanol solvent. After extensive rinsing, the implant was lyophilized again and packaged for ethylene oxide (EO) sterilization.

### 4.7. Method of Use of the Implant

After debridement of the wound site, the size of the defect was measured, and the implant was cut according to the defect size. Following insertion of the implant, the implant will come into contact with bodily fluids, such as blood, bone marrow, and extracellular fluid. These fluids will hydrate the implant, resulting in its expansion such that it fits tightly in the defect site. Additional fluid, such as sterile saline, can be used to facilitate implant hydration. In this way, the implant becomes securely anchored. If needed, a biological membrane, Chondral Guide (Geistlich, Swiss), can be used to cover the implant and sutured onto the native cartilage tissue to further secure the implant from potential shear stresses.

### Example 5. Method of Engineering ECM Implant for Small Articular Cartilage Defect Repair and Method of Use of the Implant

### 5.1. Method of Preparing the Implant

A method of preparing the implant is identical to that described in Example 4.

Remove a circular cylindrical implant from the assembled implant material from Example 4.6. and insert the cylindrical implant into the delivery device described in FIG. 3.

### 5.2. Method of Use of the Implant

Upon completion of preparing the wound site, select the appropriate size of the implant that has already been loaded into the delivery device as described in FIG. 3. The implant was delivered to the wound site according to the method described in the section of Brief Description of the Drawings using a delivery vehicle.

### Example 6. In Vitro Mechanical Testing of the Implant Prototypes

### 6.1. Tensile Testing

Strips of scaffold implant matrices of dimension 1 cm×3 cm×1 cm (W×L×H) were cut from Example 4. Samples are tested using a Chatillon mechanical tester CS2-225 (AMETEK, Berwyn). The implant strip was clamped along its long axis via the sample clamps. Samples were loaded under tension until failure. The ultimate tensile strength and the tensile modulus were calculated.

### 6.2. Compression Testing

Compression was also tested using a Chatillon CS2-225 (AMETEK, Berwyn). For unconfined compression testing, a 1 cm×1 cm×1 cm (L×W×H) square specimen was placed between two impermeable plates. The load was then applied from the top plate to the sample to press the sample 50% of the sample thickness (50% strain) after a preload of 0.05N was met to ensure even contact with the top plate. The compression resistance strength and the compression modulus are calculated.

### 6.3. Unconfined Compression

Multi-layer matrices were hydrated in purified water at room temperature for 1 hour. The height of each matrix was measured using a caliper after the hydration period. The sample was then placed on the base plate of the Chatillon CS2-225 force tester and compressed to 10, 20, or 50% of the corresponding sample height at a rate of 0.6 mm/min. Each scaffold underwent 10%, 20%, and 50% strain with at least 1 hour hydration in purified water at RT between each compression test. Each test was performed at room temperature.

### 6.4. Planar Shear

Two blocks were first adhered to each dry, multi-layer matrix using cyanoacrylate glue. One block was adhered to the bottom of the matrix, and the other to the top of the matrix in parallel to each other. These blocks served the purpose of clamping points to the Chatillon CS2-225 force tester's attachment clamps. After the glue dried, the matrices with their glue-attached blocks were hydrated in purified water at room temperature for 1 hour. The height of each matrix was measured using a caliper after the hydration period and then compressed to 10% of the corresponding sample height. Under this compressed condition each sample was then loaded into the force tester by clamping onto the glued blocks. The force tester then pulled on the glued blocks in opposite directions at a rate of 0.6 mm/min to apply shear into the sample until complete failure of the sample. Each test was performed at room temperature.

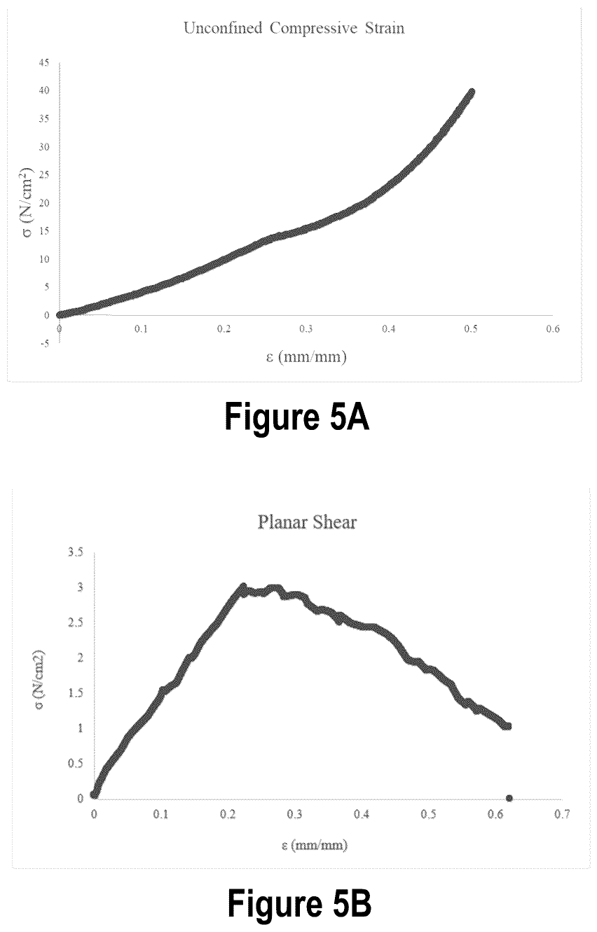

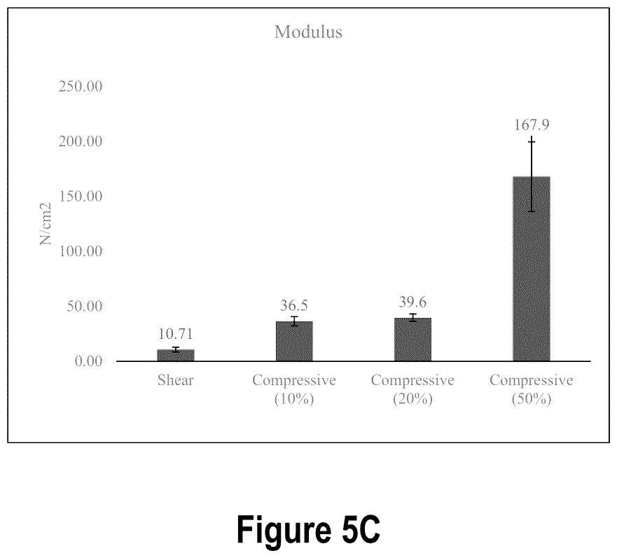

This result shows basic mechanical properties of crosslinked multi-layer matrices in the hydrated condition (FIG. 5). The multi-layer matrices experienced compressive modulus values in the order of hundreds of kilopascals (10 kilopascals=1 N/cm²) as compared to other collagen-based scaffolds reported in the literature, whose values are typically in the order of tens (or lower) of kilopascals tested in similar conditions. This result reflects the effect of the architectural design of the multi-layer matrices as well as the use of a significant solid content of insoluble collagens to provide considerable strength post-crosslinking. Integration between layers was not found to be an issue during both mechanical tests performed, especially during shear testing where no delamination occurred between layers. This critical observation indicates that the current fabrication technique is a viable method to produce a relatively stable matrix.

### Example 7. In Vitro Culture of Articular Chondrocytes in the ECM Matrices

Articular chondrocytes were isolated from finely minced articular cartilage of 4.5 months calf knee (femoral condyle) in the presence of 0.25% collagenase II enzyme. Cells were separated from the tissue and 5×10⁶ cells were injected into each matrix.

### Example 8. Cytocompatibility of the Multi-Layered Matrices

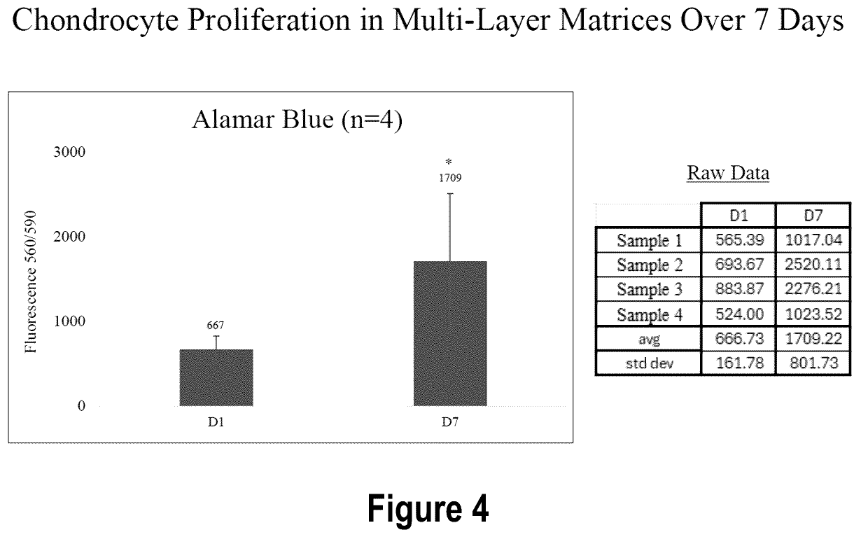

FIG. 4 shows the cytocompatibility of the multi-layered matrices after various chemical processing post-fabrication including crosslinking and post-crosslinking washes. Overall, the matrices supported the proliferation of chondrocytes, which are the primary cell population in native articular cartilage. This preliminary cell-culture experiment is believed to be the first to be performed using a matrix featuring a majority insoluble type II collagen fibers that make up several of the layers of the matrix. The solid content of type II collagen is significantly higher than what is generally reported in the scientific literature for collagen-based matrices for articular cartilage repair, which, for the most part, do not incorporate insoluble collagen let alone collagen type II in an insoluble form. As shown via Alamar Blue, the solid content of the matrices evaluated here did not hinder the diffusion of nutrients to the chondrocytes, which in turn allowed for their growth.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The present disclosure is not to be limited in scope by the specific embodiments described herein. Indeed, various modifications of the invention, in addition to those described herein, will become apparent to those skilled in the art from the foregoing description and the accompanying figures. Such modifications are intended to fall within the scope of the appended claims.

What is claimed is:

1. A biocompatible and bioresorbable extracellular matrix (ECM) implant having physically and mechanically stable stratified multi-phasic structural zones that comprise:
a surface zone of type II and type I collagen fibers aligned parallel to surface of the ECM implant, wherein the surface zone has a thickness from about 0.05 mm to about 0.5 mm;
a second zone of randomly oriented type II collagen fibers, wherein the second zone has a thickness from about 1.5 mm to about 2.0 mm;
a third zone of type II collagen fibers oriented perpendicular to surface of the ECM implant, wherein the third zone has a thickness from about 1.0 mm to about 1.5 mm;
a fourth zone of mineralizing cartilage comprising partially oriented type II collagen fibers and randomly oriented type I collagen fibers in combination with calcium-based mineral particles, wherein the fourth zone has a thickness of about 0.25 mm to about 0.75 mm; and
a mineralized zone comprising randomly oriented type I collagen fibers and calcium-based mineral particles, wherein the mineralized zone has a thickness from about 2.0 mm to about 4.0 mm.

2. The implant of claim 1, wherein the implant is adapted for repairing large articular cartilage defects, and has an area ranging from about 3 cm² to about 12 cm² and a thickness from about 0.6 mm to about 1 cm.

3. The implant of claim 1, wherein the implant is adapted for treating a small injury of an articular cartilage, and wherein the stratified multi-phasic structural zones are confined within a cell permeable biopolymeric tubular delivery vehicle having a diameter from about 0.5 cm to about 1 cm and a length from about 0.6 cm to about 1 cm to assist the delivery and insertion of the implant to a repair site of the articular cartilage.

4. The implant of claim 1, wherein the implant comprises polysaccharides.

5. The implant of claim 4, wherein the polysaccharides comprise chondroitin sulfate, keratin sulfate, hyaluronic acid, or a combination thereof.

6. The implant of claim 1, wherein the implant comprises a type III collagen, a type V collagen, a type IX collagen, a type XI collagen, or a combination thereof.

7. The implant of claim 1, wherein the implant comprises cells.

8. The implant of claim 7, wherein the cells comprise articular chondrocytes, mononuclear cells, stem cells, or a combination thereof.

9. The implant of claim 1, wherein the implant comprises bioactive molecules.

10. The implant of claim 9, wherein the bioactive molecules comprise platelet-rich plasma (PRP), growth factors, fibronectin, fibromodulin, biglycan, decorin, or a combination thereof.

11. The implant of claim 1, wherein the type II collagen fibers mixed with the type I collagen fibers at a weight ratio of from 90:10 to 60:40.

12. The implant of claim 1, wherein type I collagen fibers or type II collagens are isolated from human or animal tissues or by genetic engineering technologies.

13. The implant of claim 3, wherein the biopolymeric tubular delivery vehicle is a porous tubular biopolymer-based membrane.

14. The implant of claim 13, wherein the porous tubular biopolymer-based membrane comprises a tubular type I collagen, or a type I and type II composite collagen-based membrane.

15. The implant of claim 13, wherein the porous tubular biopolymer-based membrane is a porous tubular type I collagen-based membrane.

16. The implant of claim 15, wherein the tubular type I collagen-based membrane has a pore size of from about 20 μm to about 500 μm.

17. The implant of claim 15, wherein the tubular type I collagen-based membrane has a wall thickness of from about 0.01 mm to about 0.5 mm.

18. The implant of claim 1, wherein the stratified multi-phasic structural zones comprise from about 20% (w/w) to about 80% (w/w) of the calcium-containing mineral particles.

19. The implant of claim 1, wherein the calcium-containing mineral particles comprise synthetic and/or natural calcium-containing compounds.

20. The implant of claim 1, wherein the calcium-containing mineral particles comprise natural carbonate apatite particles.

21. A method of fabricating the implant of claim 2 for repairing large articular cartilage defects, comprising forming physically and mechanically stable stratified multi-phasic structural zones by:
preparing a fifth subchondral bone zone by mixing randomly oriented type I collagen fibers and calcium-based minerals;
preparing a fourth calcified cartilage zone by mixing randomly oriented type I fibers with calcium-based minerals and partial vertically oriented type II collagen fibers;
preparing a third deep zone by orientating the extruded type II collagen fibers in the direction orthogonal to the surface of the implant;
preparing a second middle/transitional zone by cryo-milling purified type II collagen fibers into microfibers of random fiber orientation;
preparing a surface superficial zone of type II and type I collagen-fiber sheet of random fiber orientation in two dimensions;
stabilizing the stratified multi-phasic structural zones with type I or type II collagen gelatin; and
compressing the stratified multi-phasic structural zones to a fixed height to obtain an implant.

22. The method of claim 21, further comprising crosslinking, rinsing, and lyophilizing the implant.

23. A method of fabricating the implant of claim 3 for treating a small injury of an articular cartilage, forming physically and mechanically stable stratified multi-phasic structural zones by:
preparing a fifth subchondral bone zone by mixing randomly oriented type I collagen fibers and calcium-based minerals;
preparing a fourth calcified cartilage zone by mixing randomly oriented type I fibers with calcium-based minerals and partial vertically oriented type II collagen fibers;
preparing a third deep zone by orientating the extruded type II collagen fibers in the direction orthogonal to the surface of the implant;
preparing a second middle/transitional zone by cryo-milling purified type II collagen fibers into microfibers of random fiber orientation;
preparing a surface superficial zone of type II and type I collagen-fiber sheet of random fiber orientation in two dimensions;
stabilizing the stratified multi-phasic structural zones with type I or type II collagen gelatin;
compressing the stratified multi-phasic structural zones to a fixed height to obtain an implant;
shaping the implant to a cylindrical implant of a defined diameter using a cylindrical knife cutter; and
inserting the cylindrical implant into a defined porous tubular collagen matrix.

24. An implant fabricated according to the method of claim 21.

25. An implant fabricated according to the method of claim 23.
